(19) United States
(12) Patent Application Publication
(10) Pub. No.: US 2009/0069665 A1
(43) Pub. Date: Mar. 12, 2009

(54) Automatic Lesion Correlation in Multiple MR Modalities

(75) Inventors: Gerardo Hermosillo Valadez, West Chester, PA (US); Yoshihisa Shinagawa, Downingtown, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/204,882

(22) Filed: Sep. 5, 2008

Related U.S. Application Data
(60) Provisional application No. 60/971,322, filed on Sep. 11, 2007.

(51) Int. Cl. A61B 5/055

(52) U.S. Cl. 600/410; 382/128

(57) ABSTRACT

A method for automatic correlation between multiple magnetic resonance (MR) modalities includes acquiring first MR image data form a first modality. Second MR image data is acquired from a second modality. One or more anatomical landmarks are detected within both the first and second MR image data and the first and second MR image data are automatically correlated based on the detected anatomical landmarks and interpolation using a learning deformation function. The automatic correlation is refined using a local search.

### CROSS-REFERENCE TO RELATED APPLICATION

The present application is based on provisional application Ser. No. 60/971,322 filed Sep. 11, 2007, the entire contents of which are herein incorporated by reference.

### BACKGROUND OF THE INVENTION

1. Technical Field

The present disclosure relates to lesion correlation and, more specifically, to automatic lesion correlation in multiple MR modalities.

2. Discussion of Related Art

Computer aided diagnosis (CAD) is the process of using computer vision systems to analyze medical image data and make a determination as to what regions of the image data are potentially problematic. Some CAD techniques then present these regions of suspicion to a medical professional such as a radiologist for manual review, while other CAD techniques make a preliminary determination as to the nature of the region of suspicion. For example, some CAD techniques may characterize each region of suspicion as a lesion or a non-lesion. The final results of the CAD system may then be used by the medical professional to aid in rendering a final diagnosis.

Because CAD techniques may identify lesions that may have been overlooked by a medical professional working without the aid of a CAD system, and because CAD systems can quickly direct the focus of a medical professional to the regions most likely to be of diagnostic interest, CAD systems may be highly effective in increasing the accuracy of a diagnosis and decreasing the time needed to render diagnosis. Accordingly, scarce medical resources may be used to benefit a greater number of patients with high efficiency and accuracy.

CAD techniques have been applied to the field of mammography, where low-dose x-rays are used to image a patient's breast to diagnose suspicious breast lesions. However, because mammography relies on x-ray imaging, mammography may expose a patient to potentially harmful ionizing radiation. As many patients are instructed to undergo mammography on a regular basis, the administered ionizing radiation may, over time, pose a risk to the patient. Moreover, it may be difficult to use x-rays to differentiate between different forms of masses that may be present in the patient's breast. For example, it may be difficult to distinguish between calcifications and malignant lesions.

Magnetic resonance imaging (MRI) is a medical imaging technique that uses a powerful magnetic field to image the internal structure and certain functionality of the human body. MRI is particularly suited for imaging soft tissue structures and is thus highly useful in the field of oncology for the detection of lesions.

In dynamic contrast enhanced MRI (DCE-MRI), many additional details pertaining to bodily soft tissue may be observed. These details may be used to further aid in diagnosis and treatment of detected lesions.

DCE-MRI may be performed by acquiring a sequence of MR images that span a time before magnetic contrast agents are introduced into the patient's body and a time after the magnetic contrast agents are introduced. For example, a first MR image may be acquired prior to the introduction of the magnetic contrast agents, and subsequent MR images may be taken at a rate of one image per minute for a desired length of time. By imaging the body in this way, a set of images may be acquired that illustrate how the magnetic contrast agent is absorbed and washed out from various portions of the patient's body. This absorption and washout information may be used to characterize various internal structures within the body and may provide additional diagnostic information.

Accordingly, absorption and washout information may be used to detect and characterize potential lesions from the MR image data. Other techniques may also be used to detect and characterize potential lesions within the image data. Detection and characterization of potential lesions may rely on diagnostic information collected across multiple images that are separated in time, as discussed above. Additionally, diagnostic information collected across multiple MR modalities may be considered in rendering a diagnosis.

A modality is the approach used by the MR imager to acquire data that may be used to produce the medical image. Because each modality may scan for different properties, each modality may create a distinct medical image from the same internal structure, and thus each modality may provide distinct diagnostic information, that when combined, may provide a more complete assessment of the nature of the internal structure.

Common MR modalities include the T1 relaxation modality and the T2 relaxation modality. The T1 relaxation modality examines the T1 relaxation time, also known as spin-lattice relaxation time. The T1 relaxation time characterizes the rate at which the longitudinal M_{z} component of the magnetization vector recovers. The T1 relaxation time is, more specifically, the time that it takes for the signal to recover 63% of its initial value before being flipped into the magnetic transverse plain. An image obtained using the T1 modality is considered a T1 weighted image.

Because different tissues have different T1 relaxation times, the T1 weighted image may be used to visualize the internal structure in terms of the various different types of tissue that form the structure.

The T2 relaxation modality examines the T2 relaxation time, also known as the spin-spin relaxation time. The T2 relaxation time characterizes the rate at which the M y component of the magnetization vector decays in the transverse magnetic plane. The T2 relaxation time is, more specifically, the time that it takes for the transverse signal to reach 37% of its initial value after flipping into the magnetic transverse plane. An image obtained using the T2 modality is considered a T2 weighted image.

T2 weighted images may be particularly suited for evaluating certain types of lesions such as cysts and fibro adenomas, as well as certain types of cancers. However, the T2 weighted images alone may not provide enough diagnostic information to effectively locate and characterize lesions. Accordingly, medical practitioners such as radiologists may wish to manually study both the T1 weighted image and the T2 weighted image to gather the maximum amount of diagnostic information possible. In so doing, the medical practitioner must be able to identify the same region of interest within both image modalities. This manual correlation may be difficult, time consuming, and prone to error as there are generally different structures visible from each modality.

Accordingly, because of the difficult manual correlation that is needed to combine diagnostic information associated with multiple MRI modalities, computer aided diagnostic approaches for the automatic detection of lesions in the breast have not been able to utilize multiple MR modalities.

### SUMMARY

A method for automatic correlation between multiple magnetic resonance (MR) modalities includes acquiring first MR image data form a first modality. Second MR image data is acquired from a second modality. One or more anatomical landmarks are detected within both the first and second MR image data. The first and second MR image data are automatically correlated based on the detected anatomical landmarks and interpolation using a learning deformation function.

The learning deformation function may be generated by machine learning using a plurality of sets of manually correlated images from the first and second modalities as training data. The first modality is a T1 relaxation modality and the second modality is a T2 relaxation modality.

The image data of a particular location from the first MR image may be combined with correlated image data of the particular location from the second MR image data and the combined image data may be used to determine whether the particular location is at an increased risk of being a lesion.

Image data of a region of suspicion from the first MR image may be combined with correlated image data of the region of suspicion from the second MR image data and the combined image data may be used to determine whether the region of suspicion is a lesion or a false positive.

The automatic correlation may be refined by a local search. The local search may be based on one or more of curvature, volume, or local correlation.

The first and second MR image data are acquired as part of a dynamic contrast enhanced MRI. The first and second MR image data may include a patient's breast.

A method for automatically detecting breast lesions includes receiving a dynamic contrast enhanced magnetic resonance image (DCE-MRI) of a patient's breast including image data from a first MR modality and image data of a second MR modality. One or more anatomical landmarks are detected within both the first and second MR image data. The first and second MR image data are automatically correlated based on the detected anatomical landmarks and interpolation using a learning deformation function. Image data of a particular location from the first MR image is combined with correlated image data of the particular location from the second MR image data. The combined image data is used to determine whether the particular location is at an increased risk of being a lesion.

The learning deformation function may be generated by machine learning using a plurality of sets of manually correlated images from the first and second modalities as training data.

The first modality may be a T1 relaxation modality and the second modality may be a T2 relaxation modality.

The automatic correlation may be refined by a local search prior to combining the image data and determining whether the particular location is at an increased risk of being a lesion.

The local search may be based on one or more of curvature, volume, or local correlation.

A computer system includes a processor and a program storage device readable by the computer system, embodying a program of instructions executable by the processor to perform method steps for automatic correlation between multiple magnetic resonance (MR) modalities. The method includes acquiring first MR image data form a first modality including a patient's breast. Second MR image data is acquired from a second modality including a patient's breast. One or more anatomical landmarks are detected within both the first and second MR image data. The first and second MR image data are automatically correlated based on the detected anatomical landmarks and interpolation using a learning deformation function. The automatic correlation is refined using a local search.

The learning deformation function may be generated by machine learning using a plurality of sets of manually correlated images from the first and second modalities as training data.

The first modality may be a T1 relaxation modality and the second modality may be a T2 relaxation modality.

Image data of a particular location from the first MR image may be combined with correlated image data of the particular location from the second MR image data and the combined image data may be used to determine whether the particular location is at an increased risk of being a lesion.

Image data of a region of suspicion from the first MR image may be combined with correlated image data of the region of suspicion from the second MR image data and the combined image data may be used to determine whether the region of suspicion is a lesion or a false positive.

The local search may be based on one or more of curvatures volumes or local correlation.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a flow chart illustrating a method for imaging a patient's breast using DCE-MRI and rendering a computer-aided diagnosis according to an exemplary embodiment of the present invention;

FIG. 2 is a set of graphs illustrating a correspondence between absorption and washout profiles for various BIRADS classifications according to an exemplary embodiment of the present invention;

FIG. 3 is a flow chart illustrating a method for automatically combining multiple MR modalities in the detection and characterization of regions of suspicion according to exemplary embodiments of the present invention;

FIG. 4A is a flow chart illustrating an offline process for establishing a deformation model using machine learning according to an exemplary embodiment of the present invention;

FIG. 4B is a flow chart illustrating an inline process for performing automatic correlation using the previously established deformation model according to an exemplary embodiment of the present invention; and

FIG. 5 shows an example of a computer system capable of implementing the method and apparatus according to embodiments of the present disclosure.

### DETAILED DESCRIPTION OF THE DRAWINGS

In describing exemplary embodiments of the present disclosure illustrated in the drawings, specific terminology is employed for sake of clarity. However, the present disclosure is not intended to be limited to the specific terminology so selected, and it is to be understood that each specific element includes all technical equivalents which operate in a similar manner.

Exemplary embodiments of the present invention seek to image a patient's breast using DCE-MRI techniques and then perform CAD to identify regions of suspicion that are more likely to be malignant breast lesions. By utilizing DCE-MRI rather than mammography, additional data pertaining to contrast absorption and washout may be used to accurately distinguish between benign and malignant breast masses.

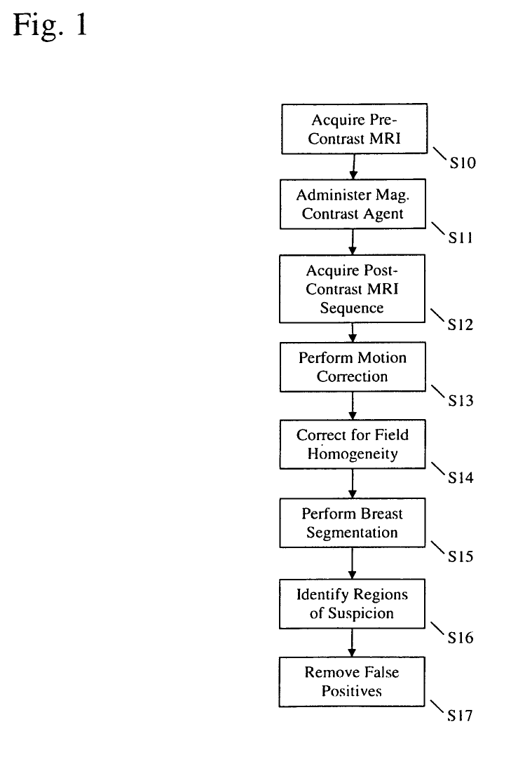

FIG. 1 is a flow chart illustrating a method for imaging a patient's breast using DCE-MRI and rendering a computer-aided diagnosis according to an exemplary embodiment of the present invention. First, a pre-contrast MRI is acquired (Step S10). The pre-contrast MRI may include an MR image taken of the patient before the magnetic contrast agent has been administered. The pre-contrast MRI may include one or more modalities. For example, both T1 and T2 relaxation modalities may be acquired.

Next, with the patient remaining as still as possible, the magnetic contrast agent may be administered (Step S11). The magnetic contrast agent may be a paramagnetic agent, for example, a gadolinium compound. The agent may be administered orally, intravenously, or by another means. The magnetic contrast agent may be selected for its ability to appear extremely bright when imaged in the T1 modality. By injecting the magnetic contrast agent into the patient's blood, vascular tissue may be highly visible in the MRI. Because malignant tumors tend to be highly vascularized, the use of the magnetic contrast agent may be highly effective for identifying regions suspected of being lesions.

Moreover, additional information may be gleamed by analyzing the way in which a region absorbs and washes out the magnetic contrast agent. For this reason, a sequence of post-contrast MR images may be acquired (Step S12). The sequence may be acquired at regular intervals in time, for example, a new image may be acquired every minute. The sequence of post-contrast MR images may include the T1 relaxation modality that is well suited for monitoring the absorption and washout of the magnetic contrast agent. For these images, acquisition of the T2 relaxation modality is not necessary.

As discussed above, the patient may be instructed to remain as still as possible throughout the entire image acquisition sequence. Despite these instructions, the patient will most likely move somewhat from image to image. Accordingly, before regions of suspicion are identified (Step S16), motion correction may be performed on the images (Step S13).

Because MR images are acquired using a powerful magnetic field, subtle inhomogeneity in the magnetic field may have an impact on the image quality and may lead to the introduction of artifacts. Additionally, the level of enhancement in the post-contrast image sequence may be affected. Also, segmentation of the breast may be impeded by the inhomogeneity, as in segmentation, it is often assumed that a particular organ appears homogeneously. Accordingly, the effects of the inhomogeneous magnetic field may be corrected for within all of the acquired MR images (Step S14).

The order in which motion correction (Step S13) and inhomogeneity correction (Step S14) are performed on the MR images is not critical. All that is required is that these steps be performed after image acquisitions for each given image, and prior to segmentation (Step S15). These corrective steps may be performed for each image after each image is acquired or for all images after all images have been acquired.

After the corrective steps (Steps S13 and S14) have been performed, breast segmentation may be performed (Step S15). Segmentation is the process of determining the contour delineating a region of interest from the remainder of the image. In making this determination, edge information and shape information may be considered.

Edge information pertains to the image intensity changes between the interior and exterior of the contour. Shape information pertains to the probable shape of the contour given the nature of the region of interest being segmented. Some techniques for segmentation such as the classical watershed transformation rely entirely on edge information. Examples of this technique may be found in L. Vincent and P. Soille, “Watersheds in digital spaces: An efficient algorithm based immersion simulations” IEEE Trans. PAMI, 13(6):583-589, 1991, which is incorporated by reference. Other techniques for segmentation rely entirely on shape information. For example, in M. Kass, A. Witkin, and D. Terzopoulous, “Snakes—Active contour models” Int J. Comp Vis, 1(4): 321-331, 1987, which is incorporated by reference, a calculated internal energy of the curvature is regarded as a shape prior although its weight is hard-coded and not learned through training. In A. Tsai, A. Yezzi, W. Wells, C. Tempany, D. Tucker, A. Fan, and W. E. Grimson, “A shape-based approach to the segmentation of medical imagery using level sets” IEEE Trans. Medical Imaging, 22(2): 137-154, 2003, which is incorporated by reference, the shape prior of signed distance representations called eigenshapes is extracted by Principal Component Analysis (PCA). When the boundary of an object is unclear and/or noisy, the shape prior is used to obtain plausible delineation.

When searching for lesions in the breast using DCE-MRI, internal structures such as the pectoral muscles that are highly vascularized may light up with the application of the magnetic contrast agent. Thus, the pectoral muscles, and other such structures may make location of breast lesions more difficult. Accordingly, by performing accurate segmentation, vascularized structures that are not associated with the breast tissue may be removed from consideration thereby facilitating fast and accurate detection of breast lesions.

After segmentation has been performed (Step S15), the breast tissue may be isolated and regions of suspicion may be automatically identified within the breast tissue region (Step S16). A region of suspicion is a structure that has been determined to exhibit one or more properties that make it more likely to be a breast lesion than the regions of the breast tissue that are not determined to be regions of suspicion. Detection of the region of suspicion may be performed by systematically analyzing a neighborhood of voxels around each voxel of the image data to determine whether or not the voxel should be considered part of a region of suspicion. This determination may be made based on the acquired pre-contrast MR image as well as the post-contrast MR image. Such factors as size and shape may be considered.

Moreover, the absorption and washout profile of a given region may be used to determine whether the region is suspicious. This is because malignant tumors tend to show a rapid absorption followed by a rapid washout. This and other absorption and washout profiles can provide significant diagnostic information.

As discussed above, information gleamed from the T1 and T2 MR modalities may be used to determine whether the region is suspicious, especially when the T1 data is correlated with the T2 data. Exemplary embodiments of the present invention automatically correlate T1 and T2 weighted images and use the diagnostic information from both modalities to determine whether a region is suspicious.

Breast imaging reporting and data systems (BIRADS) is a system that has been designed to classify regions of suspicion that have been manually detected using conventional breast lesion detection techniques such as mammography and breast ultrasound. Under this approach, there are six categories of suspicious regions. Category 0 indicates an incomplete assessment. If there is insufficient data to accurately characterize a region, the region may be assigned to category 0. A classification as category 0 generally implies that further imaging is necessary. Category 1 indicates normal healthy breast tissue. Category 2 indicates benign or negative. In this category, any detected masses such as cysts or fibroadenomas are determined to be benign. Category 3 indicates that a region is probably benign, but additional monitoring is recommended. Category 4 indicates a possible malignancy. In this category, there are suspicious lesions, masses or calcifications and a biopsy is recommended. Category 5 indicates that there are masses with an appearance of cancer and biopsy is necessary to complete the diagnosis. Category 6 is a malignancy that has been confirmed through biopsy.

Exemplary embodiments of the present invention may be able to characterize a given region according to the above BIRADS classifications based on the DCE-MRI data and/or the T1 and T2 registered image data. To perform this categorization, the absorption and washout profile, as gathered from the post-contrast MRI sequence, for each given region may be compared against a predetermined understanding of absorption and washout profiles.

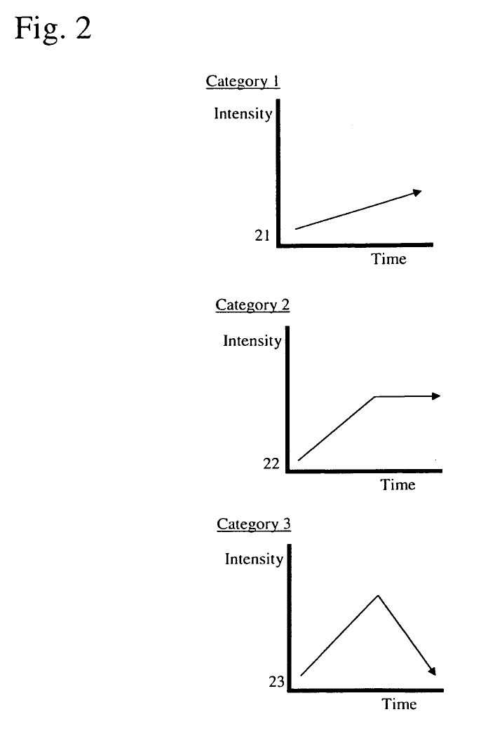

FIG. 2 is a set of graphs illustrating a correspondence between absorption and washout profiles for various BRADS classifications according to an exemplary embodiment of the present invention. In the first graph 21, the T1 intensity is shown to increase over time with little to no decrease during the observed period. This behavior may correspond to a gradual or moderate absorption with a slow washout. This may be characteristic of normal breast tissue and accordingly, regions exhibiting this profile may be classified as category 1.

In the next graph 22, the T1 intensity is shown to increase moderately and then substantially plateau. This behavior may correspond to a moderate to rapid absorption followed by a slow washout. This may characterize normal breast tissue or a benign mass and accordingly, regions exhibiting this profile may be classified as category 2.

In the next graph 23, the T1 intensity is shown to increase rapidly and then decrease rapidly. This behavior may correspond to a rapid absorption followed by a rapid washout. While this behavior may not establish a malignancy, it may raise enough suspicion to warrant a biopsy, accordingly, regions exhibiting this profile may be classified as category 3.

Other absorption and washout profiles may be similarly established for other BIRAD categories. In this way, DCE-MRI data may be used to characterize a given region according to the BIRADS classifications. This and potentially other criteria, such as size and shape, may thus be used to identify regions of suspicion (Step S16).

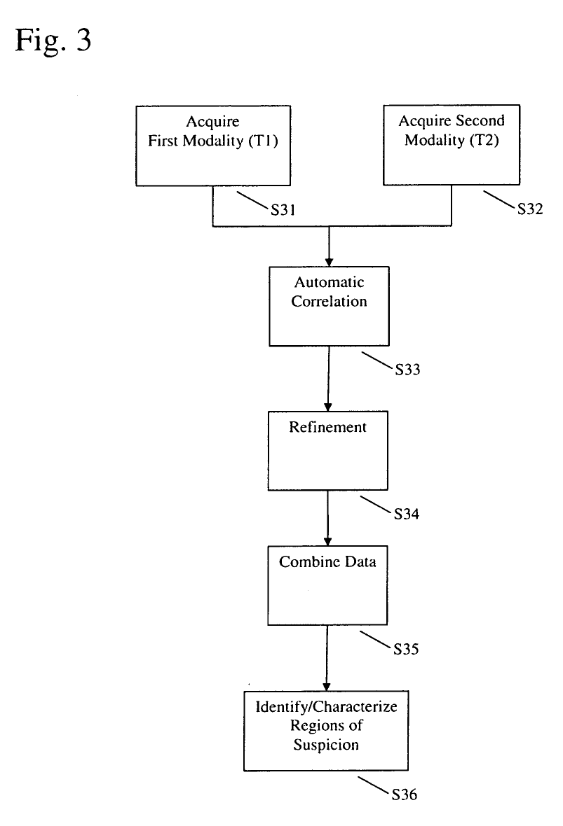

FIG. 3, discussed in detail below, illustrates how T1 and T2 image data may be automatically correlated and analyzed to identify and characterize regions of suspicion. These approaches may be used in addition to or instead of absorption and washout profiles to identify the regions of suspicion (Step S116).

After regions of suspicion have been identified, false positives may be removed (Step S117). As described above, artifacts such as motion compensation artifacts, artifacts cause by magnetic field inhomogeneity, and other artifacts, may lead to the inclusion of one or more false positives. Exemplary embodiments of the present invention and/or conventional approaches may be used to reduce the number of regions of suspicion that have been identified due to an artifact, and thus false positives may be removed. Removal of false positives may be performed by systematically reviewing each region of suspicion multiple times, each time for the purposes of removing a particular type of false positive. Each particular type of false positive may be removed using an approach specifically tailored to the characteristics of that form of false positive. Examples of such approaches are discussed in detail below.

After false positives have been removed (Step S17), the remaining regions of suspicion may be presented to the medical practitioner for further review and consideration. For example, the remaining regions of interest may be highlighted within a representation of the medical image data. Quantitative data such as size and shape measurements and/or BIRADS classifications may be presented to the medical practitioner along with the highlighted image data. The presented data may then be used to determine a further course of testing or treatment. For example, the medical practitioner may use the presented data to order a biopsy or refer the patient to an oncologist for treatment.

As discussed above, exemplary embodiments of the present invention may automatically correlate multiple MR modalities in identifying and characterizing regions of suspicion. By providing automatic correlation that is fast, efficient and accurate, information provided by multiple MR modalities may be used as part of a computer aided diagnostic system.

FIG. 3 is a flow chart illustrating a method for automatically combining multiple MR modalities in the detection and characterization of regions of suspicion according to exemplary embodiments of the present invention. Medical image data may be acquired with a first MR modality (Step S31). The first MR modality may be a T1 relaxation modality or any other available MR modality. Medical image data may also be acquired with a second MR modality (Step S32). The second MR modality may be a T2 relaxation modality or any other available MR modality. The second modality is a modality that is different from the first modality. The order in which the two modalities are used to acquire medical image data is not important, it is only necessary that each modality be used to image a region that includes the region of interest that is being analyzed, that region of interest being described herein as the breast by way of example.

After the medical image data has been acquired in the first and second modality (Steps S31 and S32), the two image modalities may be automatically correlated (Step S33). Automatic correlation may be based on a combination of detection of anatomical landmarks, for example, blood vessels and bifurcations thereof, and a learned model of deformation.

The automatic correlation of Step S33 may be a course registration, and the course registration may be followed by a refined local search that is based on image features such as curvature, volume, local correlation, etc. (Step S34).

Detection of anatomical landmarks may contribute to generating the course correlation by automatically detecting certain anatomical landmarks such as the nipple, the tip of the ribs, the interstice between the sternum and the manubrium in both modalities. The approximate coordinates of any given location on either modality may be determined in terms of their spatial relationship to the detected landmarks. Accordingly, a region of suspicion may be coarsely matched between the first modality and the second modality by its location in each modality relative to the detected landmarks.

By using landmarks as discussed above, the approximate location of a lesion may be found in each modality if it is in the vicinity of a landmark, but when the lesion is between landmarks, interpolation may be used to enable location for the purposes of course matching. The simplest form of interpolation may be to assume linearity between landmarks. However, this approach may be overly rigid. Accordingly, exemplary embodiments of the present invention may use a learned model of deformation to interpolate the location of regions of suspicion based on the detected landmarks so that the same regions of suspicion may be accurately registered between modalities.

Accordingly, the learned model of deformation may provide for interpolation between the identified landmarks. According to this approach, while off-line (in a training mode), training data in the form of pairs of T1 and T2 weighted MR images that have been manually co-registered by an expert may be provided to a learning algorithm. The learning algorithm may establish deformation model parameters that relate the T1 and T2 weighted images to one another. The deformation model parameters may be optimized for all training data so that a nearly optimal interpolation between the landmarks may be achieved.

When in-line (in diagnostic use), the learned interpolation may then be used to co-register the T1 and T2 weighted images based on the detected anatomical landmarks to form the rough correlation (Step S33). The rough correlation may then be refined (Step S34). Refinement may be performed, for example, as discussed above, based on image features such as curvature, volume, local correlation, etc. (Step S34). This may entail searching for minor structural features detected in one modality for their respective location in the other modality using the rough correlation as a starting point. Once these features are found, the rough correlation may be modified accordingly.

Minor features may be features that would be difficult to detect without a rough correlation, for example, because similar structures may appear in different locations throughout the images. However, once a course registration is determined, the minor features can significantly increase the quality of the registration. The minor features stand in contrast to the anatomical landmarks that are sufficiently distinct to be located without the aid of a rough correlation.

After the correlation has been refined, the resulting fine correlation may be used to combine data relating to a particular region from the first modality with data relating to the same region from the second modality (Step S35). The combined modality data may then be used to identify a region of suspicion, as is described above with reference to Step S16 or to determine that a previously identified region of suspicion is in fact a false positive, as is described above with reference to Step S17.

Accordingly exemplary embodiments of the present invention provide for a two-part process for performing automatic correlation. In the first part, the deformation model may be established with the use of a learning approach, and in the second part, automatic correlation is performed using the previously learned deformation model. Here, the first part is considered an offline process and the second part is considered an inline process.

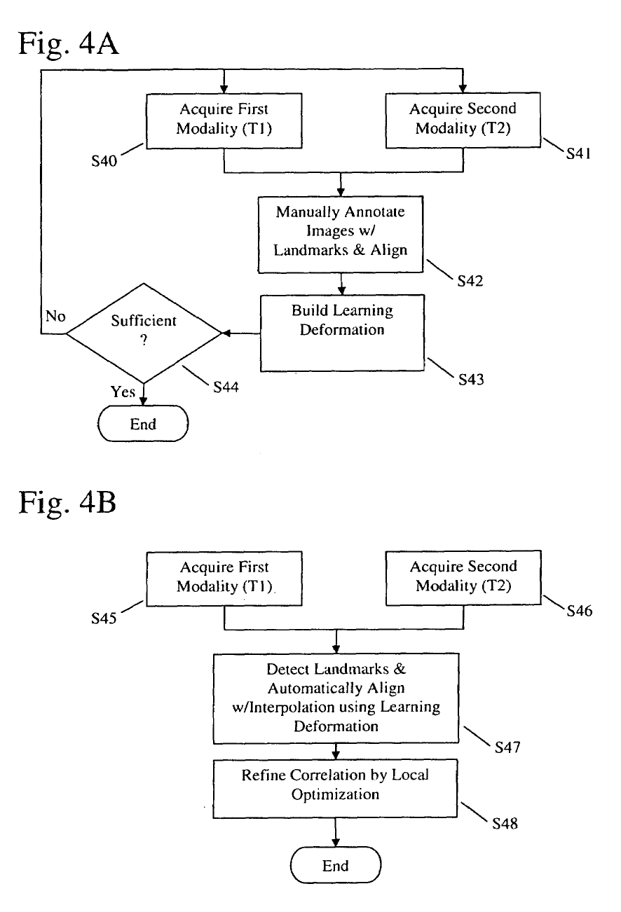

FIG. 4A is a flow chart illustrating an offline process for establishing a deformation model using machine learning according to an exemplary embodiment of the present invention. FIG. 4B is a flow chart illustrating an inline process for performing automatic correlation using the previously established deformation model according to an exemplary embodiment of the present invention.

With respect to FIG. 4A, machine learning may begin with the acquisition of a pair of first and second MR modalities form a first subject (Steps S40 and S41). Acquisition may be performed directly from an MR scanner, or the pair of medical images may be retrieved from a database of previously acquired medical images. The first and second modalities may include the T1 and T2 modalities; however, other modalities may be used. It is important that the two modalities used during the offline learning stage be the same two modalities used during the clinical inline stage.

A trained medical professional such as a radiologist may then examine the acquired medical images and annotate, on each image, the location of key anatomical landmarks (Step S42). In this step, the medical professional may also manually adjust interpolation parameters to obtain optimal alignment between the two modalities. Next, a learning algorithm may be used to process the manually adjusted image parameters and learn image patters that may be used to automatically detect the same anatomical landmarks in subsequent medical images and learn the distribution of interpolation parameters (Step S43). In this step, the learning deformation may be established.

It may then be determined whether a sufficient number of sets of medical images have been processed (Step S44). If the number of sets of medical images are not sufficient and additional sets are needed (No, Step S44) then additional first and second modality medical images may be acquired (Steps S40 and S41). If the number of sets of medical images are sufficient and no additional sets are needed (Yes, Step S44) then the learning deformation may be complete. It may be determined that no additional sets are needed when subsequent sets no longer have a significant impact on the interpolation parameters of the learning deformation.

With respect to FIG. 4B, after the learning deformation has been optimized in the offline process discussed above with respect to FIG. 4A, an inline process may be performed in the clinical setting to automatically correlate multiple modalities of MR images for computer aided diagnosis. According to this process, a pair of first and second MR modalities may be acquired from a subject (Steps S45 and S46). Then, the two images may be automatically aligned by detecting the anatomical landmarks within the two images and performing plausible interpolation based on the learned deformation (Step S47). During this step, previously detected lesions in each of the modalities may be roughly correlated based on the alignment. Finally, the roughly correlated lesions between the two modalities may be refined using local optimization around the predicted lesion locations of the rough correlation (Step S48). Here, optimization may be performed using local correlation.

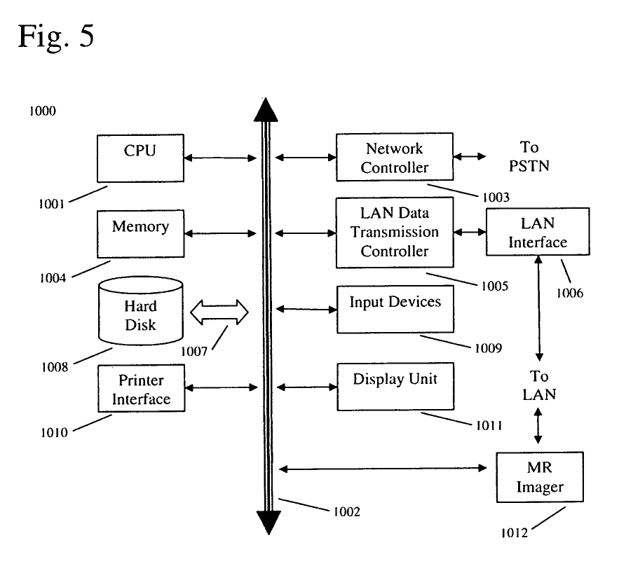

FIG. 5 shows an example of a computer system which may implement a method and system of the present disclosure. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1001, random access memory (RAM) 1004, a printer interface 1010, a display unit 1011, a local area network (LAN) data transmission controller 1005, a LAN interface 1006, a network controller 1003, an internal bus 1002, and one or more input devices 1009, for example, a keyboard, mouse etc. As shown, the system 1000 may be connected to a data storage device, for example, a hard disk, 1008 via a link 1007. A MR imager 1012 may be connected to the internal bus 1002 via an external bus (not shown) or over a local area network.

Exemplary embodiments described herein are illustrative, and many variations can be introduced without departing from the spirit of the disclosure or from the scope of the appended claims. For example, elements and/or features of different exemplary embodiments may be combined with each other and or substituted for each other within the scope of this disclosure and appended claims.

What is claimed is:

1. A method for automatic correlation between multiple magnetic resonance (MR) modalities, comprising:
acquiring first MR image data form a first modality;
acquiring second MR image data from a second modality;
detecting one or more anatomical landmarks within both the first and second MR image data;
automatically correlating the first and second MR image data based on the detected anatomical landmarks and interpolation using a learning deformation function.

2. The method of claim 1, wherein the learning deformation function is generated by machine learning using a plurality of sets of manually correlated images from the first and second modalities as training data.

3. The method of claim 1, wherein the first modality is a T1 relaxation modality and the second modality is a T2 relaxation modality.

4. The method of claim 1, further comprising:
combining image data of a particular location from the first MR image with correlated image data of the particular location from the second MR image data; and
using the combined image data to determine whether the particular location is at an increased risk of being a lesion.

5. The method of claim 1, further comprising:
combining image data of a region of suspicion from the first MR image with correlated image data of the region of suspicion from the second MR image data; and
using the combined image data to determine whether the region of suspicion is a lesion or a false positive.

6. The method of claim 1, wherein the automatic correlation is refined by a local search.

7. The method of claim 6, wherein the local search is based on one or more of curvature, volume, or local correlation.

8. The method of claim 1, wherein the first and second MR image data are acquired as part of a dynamic contrast enhanced MRI.

9. The method of claim 1, wherein the first and second MR image data include a patient's breast.

10. A method for automatically detecting breast lesions, comprising:
receiving a dynamic contrast enhanced magnetic resonance image (DCE-MRI) of a patient's breast including image data from a first MR modality and image data of a second MR modality;
detecting one or more anatomical landmarks within both the first and second MR image data;
automatically correlating the first and second MR image data based on the detected anatomical landmarks and interpolation using a learning deformation function;
combining image data of a particular location from the first MR image with correlated image data of the particular location from the second MR image data; and
using the combined image data to determine whether the particular location is at an increased risk of being a lesion.

11. The method of claim 10, wherein the learning deformation function is generated by machine learning using a plurality of sets of manually correlated images from the first and second modalities as training data.

12. The method of claim 10, wherein the first modality is a T1 relaxation modality and the second modality is a T2 relaxation modality.

13. The method of claim 10 wherein the automatic correlation is refined by a local search prior to combining the image data and determining whether the particular location is at an increased risk of being a lesion.

14. The method of claim 13, wherein the local search is based on one or more of curvature, volume, or local correlation.

15. A computer system comprising:
a processor; and
a program storage device readable by the computer system, embodying a program of instructions executable by the processor to perform method steps for automatic correlation between multiple magnetic resonance (MR) modalities, the method comprising:
acquiring first MR image data form a first modality including a patient's breast;
acquiring second MR image data from a second modality including a patient's breast;
detecting one or more anatomical landmarks within both the first and second MR image data;
automatically correlating the first and second MR image data based on the detected anatomical landmarks and interpolation using a learning deformation function; and
refining the automatic correlation using a local search.

16. The computer system of claim 15, wherein the learning deformation function is generated by machine learning using a plurality of sets of manually correlated images from the first and second modalities as training data.

17. The computer system of claim 15, wherein the first modality is a T1 relaxation modality and the second modality is a T2 relaxation modality.

18. The computer system of claim 15, further comprising:
combining image data of a particular location from the first MR image with correlated image data of the particular location from the second MR image data; and
using the combined image data to determine whether the particular location is at an increased risk of being a lesion.

19. The computer system of claim 15, further comprising:
combining image data of a region of suspicion from the first MR image with correlated image data of the region of suspicion from the second MR image data; and
using the combined image data to determine whether the region of suspicion is a lesion or a false positive.

20. The computer system of claim 15, wherein the local search is based on one or more of curvature, volume, or local correlation.
